(19) United States
(12) Patent Application Publication
(10) Pub. No.: US 2015/0290248 A1
(43) Pub. Date: Oct. 15, 2015

(54) FIBROUS COMPONENT FOR HEALTH, PERFORMANCE, AND AESTHETIC TREATMENT

(71) Applicant: NANOFIBER HEALTH, INC., Hilo, HI (US)

(72) Inventor: TIMM PEDDIE, Atlanta, GA (US)

(73) Assignee: NANOFIBER HEALTH, INC., Hilo, HI (US)

(21) Appl. No.: 14/615,501

(22) Filed: Feb. 6, 2015

Related U.S. Application Data
(60) Provisional application No. 61/977,775, filed on Apr. 10, 2014.

(51) Int. Cl. A61K 35/28; A61K 31/785; A61K 45/06; A61K 31/74

(52) U.S. Cl. CPC A61K 35/28; A61K 31/74; A61K 31/785; A61K 45/06

(57) ABSTRACT

Described herein are methods for initiating or enhancing tissue generation and tissue regeneration in a subject. The method generally involves applying at a desired site in the subject by a non-surgical technique a composition composed of a fibrous component, wherein the fibrous component comprises fibers having a diameter between 100 nm and 10 μm. The fibrous component can be used alone or in combination with one or more additional agents depending upon the application. The methods described herein have numerous applications in the medical and cosmetic surgery industry.

### CROSS REFERENCE TO RELATED APPLICATION

This application claims priority upon U.S. Provisional Application Ser. No. 61/977,775 filed on Apr. 10, 2014. This application is hereby incorporated by reference in its entirety for all of its teachings.

### BACKGROUND

Health and medical professionals seek non-surgical means of promoting healing and physical modification of a body for a variety of physical ailments or aesthetic indications. The reasons are broad and growing and may include such topics as arthritis, bone fractures and imperfections, muscular, tendon, or ligament tears and separations, or other muscular and joint-related issues to aesthetic applications such as breast or lip enlargement, softening areas of wrinkles and signs of aging, whether facial or other parts of a body, plastic surgery enhancement, or other requests for physical form modification.

With the identification of stem cells, and multipotent and pluripotent cells, and function-specific or differentiated cells, health professionals may inject said cells into problematic or aesthetically desired areas. Currently, when cells get injected, however, the recipient body typically assimilates the majority of the cells, sometimes over 98%, in less than 48 hours and without qualified benefit to the recipient.

While a variety of surgical means may exist for addressing certain ailments discussed above, most health professionals are not surgeons or are limited in their vertical expertise (i.e., specializing in just one or two surgical areas). As such, the ability to use non-surgical techniques provides greater opportunities for health professionals to address certain medical and aesthetic issues in a subject. As a result, it would be highly advantageous to health professionals in the health, beauty, and medical industries to have at their disposal non-surgical techniques for addressing a number of different issues confronted by patients today.

### SUMMARY

Described herein are methods for initiating or enhancing tissue generation and tissue regeneration in a subject. The method generally involves applying at a desired site in the subject by a non-surgical technique a composition composed of a fibrous component, wherein the fibrous component comprises fibers having a diameter between 100 nm and 10 μm. The fibrous component can be used alone or in combination with one or more additional agents depending upon the application. The methods described herein have numerous applications in the medical and cosmetic surgery industry.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

Referring now to the drawings in which like reference numbers represent corresponding parts throughout:

FIG. 1 is a simplified block diagram illustrating example of implementing the present invention.

FIG. 2 shows the fibrous component prior to injection five minutes after cell incorporation and adherence of cells to the nanofibers.

FIG. 3 shows the fibrous component prior to injection thirty minutes after cell incorporation, showing the proliferation of cells through the nanofibers and illustration of cell promotion.

### DETAILED DESCRIPTION

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

A weight percent of a component, unless specifically stated to the contrary, is based on the total weight of the formulation or composition in which the component is included.

The term “initiate” with respect to tissue generation as used herein is defined as commencing tissue generation in a subject where there was no pre-existing tissue growth. Prior to the administration of the fibrous component described herein, there was no tissue generation of interest in the subject.

The term “enhance” with respect to tissue generation as used herein is defined as increasing the rate of tissue generation in a subject when the subject is administered the fibrous component when compared to the rate of tissue generation in the same subject in the absence of fibrous component.

The term “reduce” as used herein is the ability of the methods described herein to diminish one or more symptoms in a subject when administered the fibrous component compared to the same symptom(s) in the subject that was not administered the fibrous component.

The term “prevent” as used herein is the ability of the methods described herein to eliminate the onset of one or more symptoms in a subject when administered the fibrous component compared to the same symptom(s) in the subject that was not administered the fibrous component.

The term “subject” as defined herein is any organism in need of tissue generation. In one aspect, the subject is a mammal including, but not limited to, humans, domesticated animals (e.g., dogs, cats, horses), livestock (e.g., cows, pigs), and wild animals.

The term “non-surgical technique” is defined herein as any minimally-invasive technique for delivering the fibrous component to the subject. Thus, non-surgical techniques do not include incorporating allografts, autografts, implants, and medical devices into a subject, other than through needle injection, laparoscopic surgery or other minimally invasive technique using very small breaks in the skin or body cavity, as related to the invention and use of the fibrous component.

The term “tissue generation” is defined herein as the growth of new tissue or the regeneration of existing tissue using the methods described herein. Examples of tissue described herein include, but are not limited to, ocular tissue (e.g., lens, cornea, optic nerve or retina), intestinal tissue, internal organs such as the liver, kidney, spleen, pancreas, esophagus, trachea, uterus, stomach, bladder, muscles, tendons, ligaments, nerves, dura matter and other brain structures, dental structures, blood vessels, skin (e.g., dermis, epidermis, reticular region, hypodermis), cartilage, and other bodily structures. Described herein are methods for initiating or enhancing tissue generation in a subject by administering to the subject a minimally-evasive technique a composition composed of a fibrous component, wherein the fibrous component is composed of fibers having a diameter between 100 nm and 10 μm. Depending upon the mode of administration, the dimensions of the fibrous component can vary. In one aspect, when the fibrous component is injected in a subject, the dimensions of fibrous component must suitable to be injected through the needle into the subject. The needle gauge can also vary from smaller than a 34 gauge needle (inner diameter of 0.18 mm) to larger than a 14 gauge needle (2.1 mm)

The fibrous component can be composed of a variety of biocompatible materials. In one aspect, the fibrous component can be composed of a synthetic polymer. Examples of synthetic polymers useful herein include, but are not limited to, a polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polyglycerol sebacic, a polydiol citrate, a polyhydroxy butyrate, a polyhydroxy butyrate-co-β-hydroxy valerate, a polyether amide, a polydiaxanone, poly(lactic-co-glycolic acid), or any combination or derivative thereof. In one aspect, the synthetic polymer is poly(lactic-co-glycolic acid), where the amount of polylactide in the copolymer is from 50% to 95%, 60% to 90%, 70% to 85%, or 80% to 85% by weight of the copolymer and the amount of polyglycolide in the copolymer is from 5% to 50%, 10% to 40%, 15% to 30%, or 15% to 20% by weight of the copolymer.

In another embodiment, the fibrous component can be a natural biological material. Examples of such natural materials useful herein include, but are not limited to, fibronectin, collagen, elastin, laminin, tenascin, gelatin, hyaluronic acid, chitosan, or any combination thereof. In the case when the fibrous component is a natural biological material, the fibrous component can be synthesized in vitro using techniques known in the art.

The fibrous component can be prepared by techniques known in the art. In one aspect, a polymer network composed of synthetic fibers having a diameter between 100 nm and 10 μm can be produced by electrospinning, force spinning, extrusion, or other techniques known in the art. In one aspect, the fibers have a diameter of 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, or 10 μm, where any value can be a lower- or upper-endpoint of a diameter range. Depending upon the conditions used for fiber formation, the resulting fibers in the fibrous component can be aligned (i.e., the majority of the fibers are parallel with one another), non-aligned (i.e., random configuration of fibers), or a combination of both, etc. The fibrous component may be prepared from a thin sheet composed of a polymeric network. In one embodiment, the polymer network can be milled or refined such that the polymer network is broken up or otherwise reduced in size into particles (i.e., fibrous component) having a particular dimension to form the fibrous component of invention. In other embodiments, the fibrous component can be made directly with the use of a 3-D printer or other technological means of creating the fibrous component directly.

The dimensions of the fibrous component can vary depending upon the mode of administration to the subject. For example, when the fibrous component is administered to the subject via injection, the dimensions of the fibrous component can be adjusted so that the fibrous component readily passes through the needle. The gauge of the needle can vary depending upon the application. In certain aspects, needles as large as 14 gauge (1.6 mm inner diameter) can be used. In other aspects, needles as small as 34 gauge and smaller can be used. In one aspect, the fibrous component has dimensions (e.g., length, width, height) that are less than 2.00 mm In other aspects, the fibrous component has dimensions that are from 0.0001 mm to 2.0 mm, 0.1 mm to 1.5 mm, 0.2 mm to 1.0 mm, or 0.3 mm to 0.75 mm. In another aspect, the fibrous component has dimensions so that it can readiliy pass through a 14 gauge needle to a 34 gauge needle, or other smaller needle, as technologies progress.

Depending upon the selection of the polymer used to produce the fibrous component, the fibrous component can also be biodegradable. For example, poly(lactic-co-glycolic acid) is a biodegradable polymer that can degrade in vivo over several weeks.

In the case when the fibrous component is to be administered to the subject via injection, the fibrous component can be formulated in any excipient the biological system or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. For example, the pH of the composition is from about 5 to about 6, which is suitable for topical applications. Additionally, the pharmaceutical compositions can include carriers, thickeners, diluents, preservatives, surface active agents and the like in addition to the compounds described herein.

Prior to administration, a plurality of cells can be applied to and anchored to the fibrous component. Depending upon the application, the cells can further enhance tissue generation or regeneration when used in combination with the fibrous component. Examples of cells useful herein include, but are not limited to, induced or standard pluripotent cells. In one aspect, autologous or allogeneic cells such as cord blood cells, embryonic stem cells, mesenchymal cells, placental cells, bone marrow derived cells can be used herein. In another aspect, specialized cells such as hematopoietic cell, adipose cells, epithelial cells, endothelial cells, fibroblasts, chondrocytes or any combination thereof can be used herein. In one aspect, the cells are harvested from the subject prior to administration with the fibrous component using techniques known in the art.

In one aspect, the cells are undifferentiated cells such as stem cells, induced stem cells, or progenitor cells. In another aspect, the undifferentiated cells include stem cells derived from adipose tissue or bone marrow of the subject. In another aspect, the cells include specialized cells such as the ectoderm, endoderm, or mesoderm, whether keratinocytes, melanocytes, Merkel cells, chondrocytes, epithelium cells, non-epithelial blood cells, mesothelium cells, mesenchymal cells, coelomocytes, or other specialized cells.

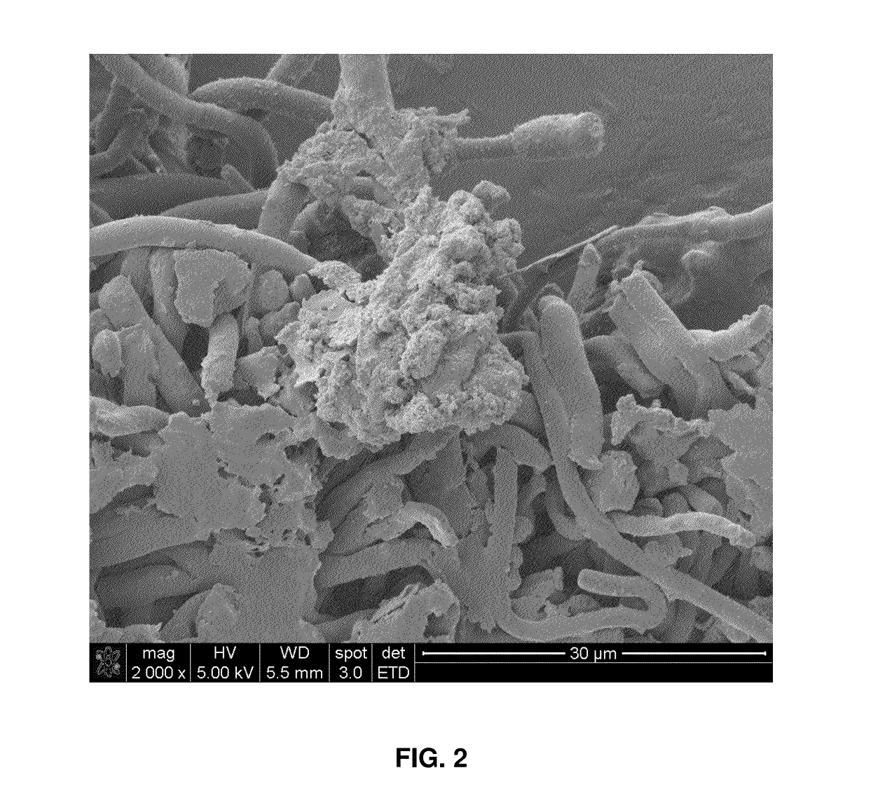

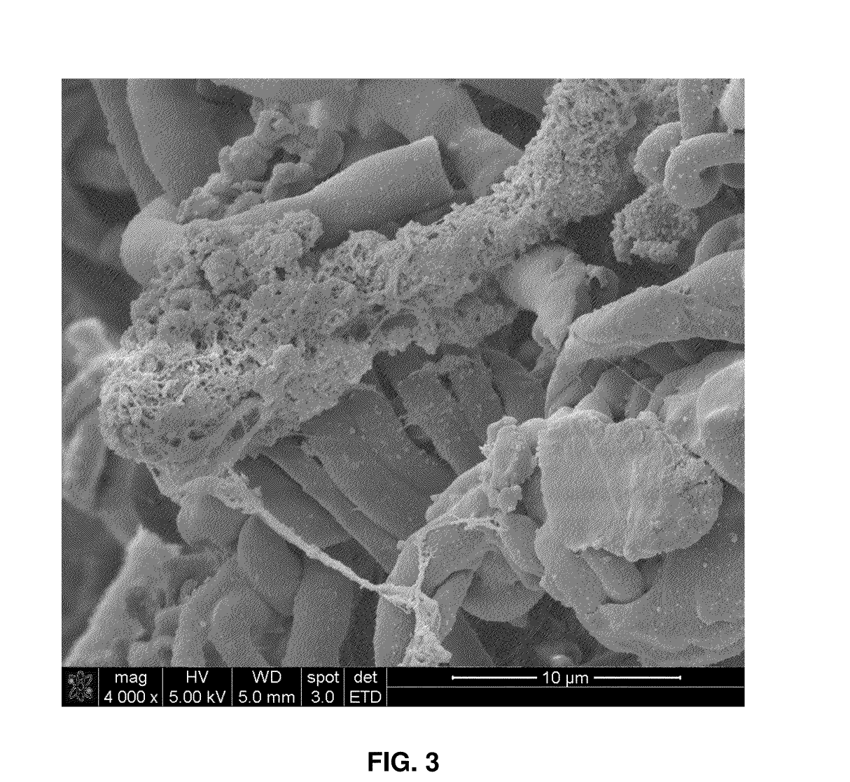

Not wishing to be bound by theory, the fibrous component serves as a scaffold so the cells can infiltrate into the fibrous component. The cells then attach to the fibrous component, offering a form of anchor whereby the fibrous component helps keep the cells alive longer as well as helping keep the cells in the desired location in the subject longer, which will also enhances tissue generation. This is depicted in FIGS. 2 and 3. In the absence of the fibrous component, after being injected into the subject, cells will be flushed out from the injection site, which significantly diminishes the regenerative tissue of the area.

In certain aspects, the cells are further activated with platelet rich plasma (PRP). Using techniques known in the art, PRP derived from the blood of the subject can be processed with the cells, which can further activate the cells with respect to tissue generation upon administration to the subject. Once isolated or prepared, add the cells to the fibrous component. Agitate the resulting composition for a sufficient time, which might typically range from 15-60 minutes, for the cells to adhere to the fibrous component. After agitation, the suspension of fibrous component and cells can be administered into the subject. PRP may also be used in combination with the fibrous component in the absence of the cells.

The fibrous component may include one or more agents to enhance the regenerative benefit. This may include one or more types of agents, such as antibiotics to help fight or prevent an infection caused by bacteria or other microorganism. Examples of additional agents useful herein include, but are not limited to, growth factors, anti-inflammatory agents, glucosamine, or chondroitin sulfate, cytokines, or any combination thereof.

In one aspect, the agent can be incorporated in the fibrous component. For example, the agent can be admixed with a solution of the polymer (e.g., synthetic polymers or natural biological materials described herein) used to produce the polymer network that ultimately leads to the fibrous component. Upon formation of the polymer network (e.g., electrospinning), the agent is dispersed throughout each of the fibers. Not wishing to be bound by theory, when the fibrous component degrades in the subject in this embodiment, the agent is released over time in the subject. In another aspect, the agent can be applied to the surface of the fibrous component. For example, the fibrous component and agent can be admixed in solution, where the agent is applied to the surface of the fibrous component. In certain embodiments, the agent can be both incorporated within as well as applied to the surface of the fibrous component.

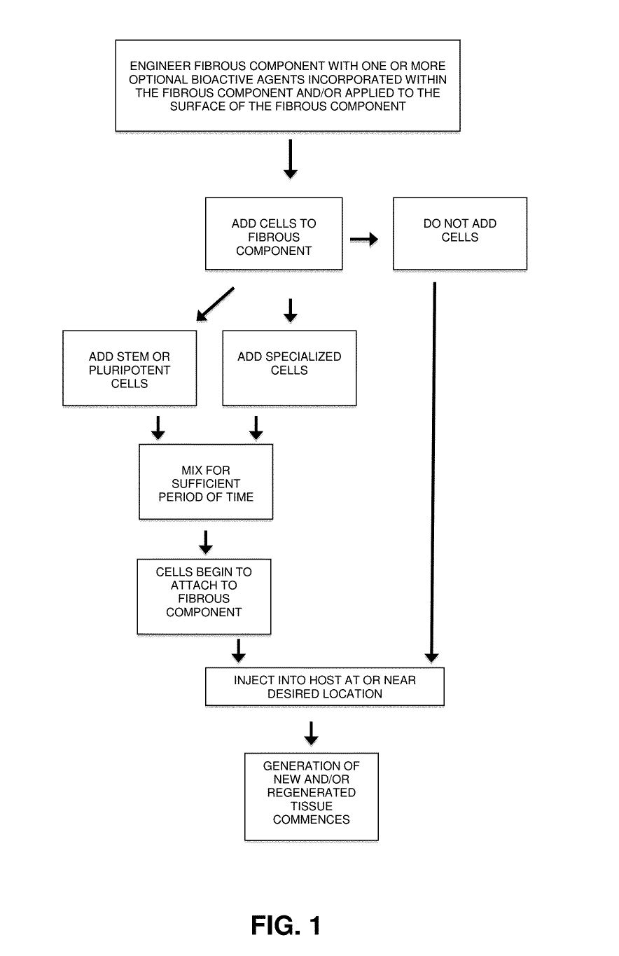

FIG. 1 is a simplified block diagram illustrating a non-limiting embodiment of the present invention for preparing and administering the fibrous components with and without cells to a subject. Here, the fibrous component with optional agent (incorporated within and/or applied to the surface of the fibrous component) is selected. Depending upon the application, cells can optionally be anchored to the fibrous component. The fibrous component with or without the cells can then be adminstered to the subject to commence tissue generation or regeneration.

Dosing with respect to the fibrous component and optional bioactive agents (e.g., cells) is dependent on severity and responsiveness of the condition to be treated, and frequency may vary with one or more doses per period, whether a period of weeks or years, as defined by the administering expert. Persons of ordinary skill can easily determine optimum dosages, dosing methodologies and repetition rates. The fibrous component can be administered multiple times to achieve sufficient tissue generation.

The methods described herein are useful in any situation where it is desirable to initiate and/or enhance tissue generation in a subject. In one aspect, the methods described herein are useful in initiating or enhancing tissue generation in a subject having arthritis. For example, when the subject has osteoarthritis, the subject has degeneration of cartilage in a joint. The fibrous component can be injected at or near the source of the arthritis. In one embodiment, the fibrous component with cells can be administered within the joint of the subject so that fibrous component comes into contact with the bones of the joint. In this aspect, new cartilage is formed on the surface of the bones present in the joint. In one aspect, the fibrous component is administered with cells derived from the subject.

Although the embodiment above addresses osteoarthritis, the fibrous component can be used to initiate or enhance tissue generation in a subject experiencing other types of arthritis including, but not limited to rheumatoid arthritis, gout, psoriatic arthritis, septic arthritis, ankylosing spondylitis (AS), Calcium Pyrophosphate Dihydrate Crystal Deposition Disease (CPPD) (Pseudo Gout), Ehlers-Danlos Syndrome (EDS), fibromyalgia (FMS), fifth disease, giant cell arteritis (GCA), juvenile arthritis, Lyme Disease, Myositis, Raynaud's Phenomenon, reactive arthritis, reflex sympathetic dystrophy syndrome (RSDS), Sjögren's syndrome, Still's Disease, systemic lupus erythematosus (Lupus), systemic lupus erythematosus (SLE), or tendinitis.

In other aspects, the methods described herein initiate or enhance muscle tissue generation in a subject. In this aspect, the methods described herein can be used to repair a muscle tear or lesion in a subject. The fibrous component can be directly administered to the subject at or near the site of the muscle tear or lesion. In one aspect, the fibrous component is administered with cells derived from the subject. In a further aspect, platelet rich plasma (PRP) derived from the subject prior admixing with the fibrous component and administration to the subject.

In one aspect, the methods described herein initiate or enhance tendon or ligament tissue generation in a subject. In this aspect, the methods described herein can be used to repair a ligament tear or lesion or a ruptured tendon in a subject. The fibrous component can be directly administered to the subject at or near the site of the muscle tear or lesion. In one aspect, the fibrous component is administered with cells derived from the subject. In a further aspect, platelet rich plasma (PRP) derived from the subject prior admixing with the fibrous component and administration to the subject. In another aspect, the fibrous component can be composed of aligned fibers so that they can be injected into the tendon using a very small needle. In this embodiment, the fibrous component can be extruded through the tendon, which will ultimately enhance the repair of the tendon.

In another aspect, the methods described herein initiate or enhance bone growth in a subject. For example, bone growth can occur at a joint of the subject where bone degeneration and degradation has occurred due to loss of cartilage in the joint.

In other aspects, the methods described herein aesthetically augment a feature of a subject. The term “aesthetically augmented” as used herein is defined as using the fibrous component to increase the number of cells and volume of extracellular matrix in the region of implementation when compared to the number of cells and volume in the region of implementation prior to the administration of the fibrous component. The augmentation in the physical feature can vary. For example, the methods described herein can augment the appearance of a variety of body parts, including lips, face, breasts, and buttocks of a subject by making the physical feature appear larger and fuller. In this aspect, the fibrous component with or without cells can be injected at the site of interest. In other aspects, the methods described herein can be used to reduce the appearance of wrinkles in a subject. Once again, the fibrous component can be injected at or near the site of the wrinkle.

The methods described herein provide an attractive approach compared to current techniques that use implantable gels. The body over time bio-absorbs the injected gel and, thus, the gel loses its effect and function over time. The fibrous component used herein provides a more permanent solution to the shortcomings of current techniques used in cosmetic augmentation and surgery.

In addition to initiating or enhancing tissue generation in a subject, the methods described herein are also effective in reducing one or more symptoms in a subject. For example, a subject with arthritis, a muscle tear, or ruptured tendon will experience significant pain and discomfort. The methods described herein initiate or enhance tissue generation in a subject, which in turn will reduce the onset of pain and other debilitating symptoms (e.g., swelling, stiffness, lameness, tenderness, inflammation, or any combination thereof) in the subject.

Additionally, the methods described herein can prevent these debilitating symptoms from developing in a subject. For example, a subject can be examined to determine if the subject will likely be afflicted with osteoarthritis. The subject may not be experiencing any symptoms but may show signs of osteoarthritis. If detected early, the fibrous component can be administered to the subject prior to significant cartilage loss and subsequent pain. In addition to preventing debilitating symptoms, the methods described herein would also avoid the need for invasive treatments such as bone fusion or joint replacement.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Example Equine Study

This example study qualifies one embodiment, showing preparation of fibrous component with stem cells derived from adipose tissue.

A subject (H-1) at level 3 to 3½ on the AAEP Lameness Scale was prepared for incision by being shaven and cleaned at the incision spot. Local anesthesia was administered and the incision site was cleaned a second time.

An incision was made and a portion of fat was removed and collected. The incision was stitched up and a fly repellent was applied.

The fat was cut into smaller pieces. Enzymes, buffer solutions, and antibiotics were added. The enzymes enabled the separation of muscle, fat, and liquid. These samples were placed in a water bath for 45 minutes. Approximately every ten minutes, the samples were shaken thoroughly.

Cell extraction solution was added to the samples to separate cells from fat. The samples with added cell extraction solution were placed back in the water bath for 15 minutes and were shaken once at the halfway point of this incubation step.

The tubes containing fat samples were placed in a centrifuge and separated into the following layers: body oils, fat, added liquids, cells, and red blood cells. Air was used to remove the red blood cells, cells, and added liquids. This solution was filtered to remove undesired components and re-centrifuged. A portion of the liquid was removed and an additional antibiotic was added.

The centrifugation and antibiotic addition was repeated an additional time, followed by filtering. Finally, the antibiotics were removed from the solution. The harvested cells were saved.

Blood from H-1 was collected in centrifuge tubes. Upon centrifugation, the blood separated into three layers: a plasma layer on top, a middle layer of white blood cells, and a bottom layer of red blood cells. The plasma layer was removed from the centrifuge tubes and centrifuged again to separate the platelets from the plasma.

Some plasma was removed from the centrifuged plasma; the rest was mixed with the platelets to form platelet rich plasma (PRP). 10 mL of activator was added to the platelets and the remaining plasma; this forms a gel (clot). The PRP samples were placed into a water bath to warm them up.

The harvested cells and PRP were mixed and subjected to LED light. After this step, the fibrous component (poly(lactic-co-glycolic acid); 82% polylactide/18% polyglycolide) was added and mixed for 15 minutes.

### Evaluation of the Fibrous Component

The suspension of fibrous component and stem cells was injected in each of H-1's front coffin joints. After 30 days, H-1 improved from 3.5 to 1.5 on the AAEP Lameness Scale.

A similar therapy was applied to a second host subject (H-2), where H-2 scored 1.5 on the AAEP Lameness Scale after an acute soft tissue injury to his left stifle. H-2 was given an injection in the medial femur-tibial joint of his left stifle. After 30 days, H-2 scored slightly less than 1.5 on the AAEP Lameness Scale with a 50% improvement in flexion.

A third host subject (H-3) with a history of chronic degenerative joint disease in her hind limbs with limited response to conventional therapies was evaluated. H-3 scored 1.5 on the AAEP Lameness Scale at a trot and 4.5 after stifle flexion. H-3 was given an injection in the medial femur-tibial joint of both stifles. After 30 days, H-3 showed 25% improvement in stifle flexion.

### II. Example Canine Study

Host subject began to slow down a bit with each progressive walk as he was aging. When the owner began to notice him limp, she took him into her veterinary doctor. However, at the time, there was no good recommendation, so she just began taking him for shorter walks, until he began to whimper at times and hobble, favoring his rear legs. This continued for over an extended period before she talked to her vet again, thinking maybe he may need to be euthanized. This time the vet offered fibrous component from invention, herein, as a specialized stem cell therapy to localize the stem cells in subject's joints. The owner volunteered the subject, where he received four injections in total; one in each of his rear hock joints, and the other two in the left and right rear stifle, where the tibia meets the femur. The stem cells were taken from the adipose fat on the subject's croup per existing documented guidelines. Following the procedure, the subject showed less movement than previously and just stayed indoors as much as possible, still hobbling after 7 days. However, by day 14, the owner visually noticed a definite improvement in not only the subject's gate but his desire to go outside. Not only did he seem to now want to go for walks, there was no noticeable prior limp and pain. After 21 days, subject ran like he did when he was in his prime, not even being prompted. At 10 weeks, subject's improvement continued to be maintained, and he has been restored to a more youthful exuberance.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A method for initiating or enhancing tissue generation or regeneration in a subject, the method comprising applying at or near a desired site in the subject by a minimally evasive technique a fibrous component, wherein the fibrous component comprises fibers having a diameter between 100 nm and 10 μm.

2. The method of claim 1, wherein the fibrous component is injected into the subject by a needle or a laparoscopic technique.

3. The method of claim 1, wherein the subject has arthritis, and the fibrous component is applied at or near the source of the arthritis.

4. The method of claim 3, wherein the arthritis is osteoarthritis, rheumatoid arthritis, gout, psoriatic arthritis, septic arthritis, ankylosing spondylitis (AS), Calcium Pyrophosphate Dihydrate Crystal Deposition Disease (CPPD) (Pseudo Gout), Ehlers-Danlos Syndrome (EDS), fibromyalgia (FMS), fifth disease, giant cell arteritis (GCA), juvenile arthritis, Lyme Disease, Myositis, Raynaud's Phenomenon, reactive arthritis, reflex sympathetic dystrophy syndrome (RSDS), Sjögren's syndrome, Still's Disease, systemic lupus erythematosus (Lupus), systemic lupus erythematosus (SLE), or tendinitis.

5. The method of claim 1, wherein the method initiates or enhances the formation of cartilage in a joint of the subject.

6. The method of claim 1, wherein the method initiates or enhances muscle tissue generation in a subject.

7. The method of claim 1, wherein the method initiates or enhances bone growth in a subject.

8. The method of claim 1, wherein the method initiates or enhances tendon tissue generation or ligament tissue generation in a subject.

9. The method of claim 1, wherein the method reduces or prevents pain, swelling, stiffness, lameness, tenderness, inflammation, or any combination thereof in the subject.

10. The method of claim 1, wherein the method aesthetically enhances a feature of the subject.

11. The method of claim 10, wherein the fibrous component is injected in the lips, breasts, buttocks, or face of the subject.

12. The method of claim 1, wherein the fibrous component is a synthetic polymer comprising a polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polyglycerol sebacic, a polydiol citrate, a polyhydroxy butyrate, a polyhydroxy butyrate-co-β-hydroxy valerate, a polyether amide, a polydiaxanone, poly(lactic-co-glycolic acid), or any combination or derivative thereof.

13. The method of claim 1, wherein the fibrous component is a natural biological material comprising fibronectin, collagen, elastin, laminin, tenascin, gelatin, growth factors, hyaluronic acid, chitosan, or any combination thereof.

14. The method of claim 1, wherein the fibrous component further comprises cells anchored to the fibrous component.

15. The method of claim 14, wherein the cells are further activated platelet rich plasma.

16. The method of claim 14, wherein the cells comprise undifferentiated cells comprising stem cells, induced stem cells, or progenitor cells.

17. The method of claim 16, wherein the undifferentiated cells comprise stem cells derived from adipose tissue or bone marrow of the subject.

18. The method of claim 14, wherein the cells comprise specialized cells comprising the ectoderm, endoderm, or mesoderm, keratinocytes, melanocytes, Merkel cells, chondrocytes, epithelium cells, non-epithelial blood cells, mesothelium cells, mesenchymal cells, or coelomocytes.

19. The method of claim 1, wherein fibrous component further comprises one or more agents comprising a growth factor, an anti-inflammatory agent, an antibiotic, glucosamine, or chondroitin sulfate, or any combination thereof, wherein the agent is incorporated in the fibrous component, on the surface of the fibrous component, or a combination thereof.

20. The method of claim 14, wherein fibrous component further comprises one or more agents comprising a growth factor, an anti-inflammatory agent, an antibiotic, glucosamine, or chondroitin sulfate, or any combination thereof, wherein the agent is incorporated in the fibrous component, on the surface of the fibrous component, or a combination thereof.

21. The method of claim 1, wherein the subject is a wild animal, a domesticated animal, livestock, or a human.
